(19) United States
(12) Patent Application Publication
(10) Pub. No.: US 2022/0260550 A1
(43) Pub. Date: Aug. 18, 2022

(54) Sequencing of Biopolymers By Motion-Controlled Electron Tunneling

(71) Applicant: Universal Sequencing Technology Corporation, Canton, MA (US)

(72) Inventors: Peiming Zhang, Gilbert, AZ (US); Ming Lei, Sharon, MA (US); Kisup Chung, Weston, MA (US); Xinyue Zhang, Norwood, MA (US); Barrett Duan, Reading, MA (US)

(21) Appl. No.: 17/627,730

(22) PCT Filed: Jul. 15, 2020
(86) PCT No.: PCT/US2020/042188

§ 371 Date: Jan. 17, 2022

Related U.S. Application Data
(60) Provisional application No. 62/874,341, filed on Jul. 15, 2019.

(51) Int. Cl. G01N 33/487; G01N 27/447; G01N 27/327

(52) U.S. Cl. CPC G01N 33/48721; G01N 27/327; G01N 27/447

(57) ABSTRACT

The present invention relates to a nanopore device with a motion control mechanism to control the speed of a polymeric molecule translocating through the nanopore for a tunneling nanogap to read out its sequences or components.

This application claims priority to U.S. Provisional application No. 62/874,341, filed Jul. 15, 2019, the contents of which are hereby incorporated herein their entirety.

### FIELD

Embodiments of the present invention are related to systems, methods, devices, and compositions of matter for identification and sequencing of biopolymers by electronic measurements. More specifically, this disclosure includes embodiments where electron tunneling nanogaps embedded in solid-state nanopores or nanoslits, which enable the detection of biopolymers electronically at a single unit or single-base level. The biopolymers are either linear, having a linear backbone, or having a linear carrier.

### BACKGROUND AND PRIOR ARTS OF THE INVENTION

With a state-of-the-art NGS sequencer, an individual human genome can be sequenced in a few days and around a thousand dollars. Compared to classical Sanger sequencing (˜800 base read length on average),¹, however, NGS reads DNA much shorter.² One of the disadvantages for the short reads is that it cannot encapsulate long blocks of repetitive sequences in the human genome, where half of the sequences are composed of repeats with the size from 1-2 bases to millions of bases.³ That poses great challenges for the assembly of human genomes. Single-molecule real-time (SMRT) sequencing, known as the third-generation sequencing (developed by Pacific Biosciences), offers sequences with an average read length of >10 kbp,⁴ allowing for the de novo assembly of large genomes, such as a gorilla's genome.⁵ However, SMRT has a much higher error rate (˜15%) for sequencing. A high sequencing coverage can overcome the error issue, but it will cost more, for example, with ˜$10,000 for a 30× human genome. For the use in clinics, ideally, the sequencing cost should be in a $100 per genome ballpark.⁶

Nanopore sequencing is a technology based on the measurement of ionic current variations, which was conceptualized three decades ago.⁷ A nanopore is an orifice with a diameter of nanometers, allowing the flow of ions across the nanopore under voltage bias. When a single-stranded DNA (ssDNA)—a polyanion—is electrophoretically translocated through the nanopore embedded in membrane that separates two chambers filled with conductive electrolytes, it blocks an ionic current transiently. Since the nucleobases have distinguishable sizes, the blockage varies as the translocation proceeds. The DNA sequences can be deduced from the ionic current fluctuations. A commercial nanopore sequencer, Min ION, has been developed by Oxford Nanopore Technologies based on protein nanopores (www.nanoporetech.com). Since there is no theoretic limit on a length of DNA for the translocation, the nanopore sequencing overcomes the short read issues related to NGS. It may be an ultimate tool for de novo sequencing and analysis of structural variations. However, the protein nanopore sequencing is difficult to achieve single-base resolution. The overall sequencing accuracy is very low (85% with a single read⁸). Gundlach and coworkers have demonstrated that the current blockage in a protein nanopore composed of Mycobacterium smegmatis porin A (known as MspA) is a collected event of four nucleotides (quadromer), and therefore there are 4⁴ (i.e., 256) possible quadromers that exert a significant number of redundant current levels.^{9,10} Because the ionic current is affected by nucleotides beyond those inside the nanopore,¹¹ even an atomically thin nanopore may not be conceivable to achieve a single nucleotide resolution for DNA sequencing.

Ventra et al. proposed to sequence DNA using a pair of electrodes separated by a distance of nanometers¹² where electrons can tunnel through such a short gap, called nanogap. Since then, the work on sequencing by nanogap electron tunneling has much progress.¹³ For the single-molecule sequencing by electron tunneling, one configuration is to embed a tunneling nanogap in a solid-state nanopore so the sequence of a single-stranded DNA can be read out sequentially by the nanogap when the molecule translocates through the nanopore. Given that the tunneling current is highly sensitive to changes in the gap size (˜an order of magnitude per Å, comparable to the distance between two adjacent bases in a single stranded DNA), the tunneling measurement has the great potential to achieve a single nucleotide resolution for DNA sequencing. It has been demonstrated that nucleoside monophosphates and oligonucleotides can generate tunneling currents in a small nanogap (<1 nm). That poses a great challenge to manufacture such a small nanogap. The prior art has provided a method to fabricate sub-3 nanometer gaps composed of palladium electrodes (U.S. Pat. No. 9,128,078). When the two electrodes are functionalizing with recognition molecules, the tunneling measurement was performed at a gap distance of ˜2.5 nm.¹⁴

### SUMMARY OF THE INVENTION

The invention provides systems, devices, and methods for electronically sequencing biopolymers, such as DNA and RNA, proteins, sugars, etc., by electron tunneling with biopolymer movement control mechanism. This disclosure demonstrates the design, fabrication, and use of this type of device for DNA sequencing in a variety of exemplary embodiments. The same devices and methods can apply to the sequencing of proteins, peptides, polysaccharides, and other synthetic chemo-functional and biofunctional polymers.

The present invention is an extension of previous applications (WO 2017/075620 and PCT/US18/32399). It uses the said devices and methods to control the motion of biopolymers in a nanopore for reading their sequences by an electron tunneling junction. The entirety of the two previous applications is included in this disclosure as a reference.

### BRIEF DESCRIPTION OF THE DRAWINGS

FIG. 1 shows a schematic illustration of motion-controlled nanopore DNA sequencing process by electron tunneling nanogap with recognition reader molecules under various embodiments: (a) a planar electron tunneling nanogap, (b) a stacked electron tunneling nanogap, (c) the process of DNA movement control and sequencing through a nanopore.

FIG. 2 shows a schematic illustration of the process of fabricating a single stacked electron tunneling nanogap embedded in a nanopore, in which the two electrodes are in different planes separated by an insulating spacer.

FIG. 3 shows a schematic illustration of the process of fabricating double-stacked electron tunneling nanogaps embedded in a nanopore, in which two pairs of electrodes are involved for the tunneling measurements.

FIG. 4 shows a schematic illustration of the process of fabricating a planar electron tunneling nanogaps embedded in a nanopore.

FIG. 5 shows a scheme for the synthesis of a xanthine-based reader molecule. molecular models of xanthine interacting with DNA nucleosides based on molecular mechanics energy minimization

FIG. 6 shows molecular models of xanthine interacting with DNA nucleosides calculated from molecular mechanics energy minimization.

FIG. 7 shows a general form of structures for the xanthine-based reader molecules.

FIG. 8 shows a general form of structure of those with a smaller size derivated from the xanthine reader molecules.

FIG. 9 shows a schematic illustration of the general structure of sample constructs.

FIG. 10 shows a process of preparing DNA constructs.

### DETAILED DESCRIPTION

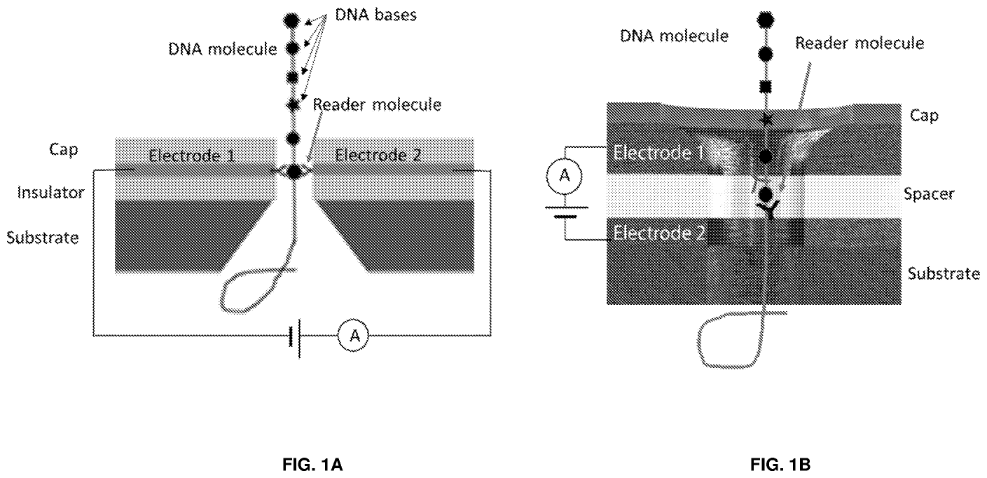

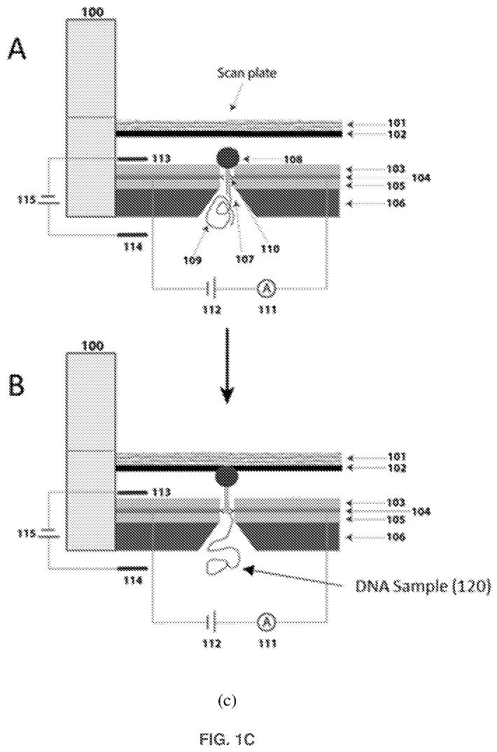

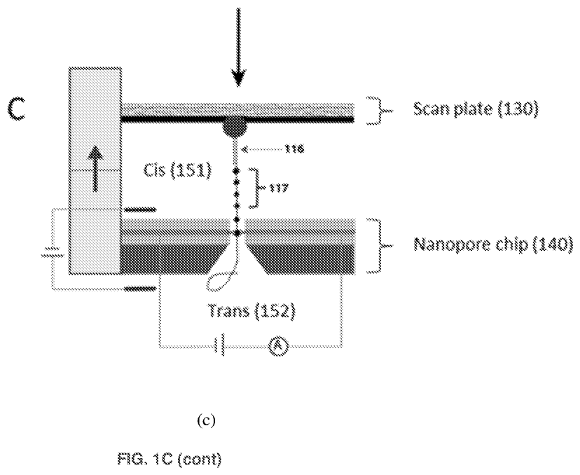

An electron tunneling nanogap, composed of a pair of electrodes separated by a distance less than 3 nanometers, can be built in a nanopore either in a planar way (separated by a gap, see FIG. 1a) or in a stacked way (separated by an insulating layer, see FIG. 1b). DNA base sensing molecules (reader molecules) are attached to the electrodes such that the molecules on opposite electrodes do not touch each other but separated by a nanogap equivalent to the size of DNA bases. When a single-stranded DNA translocates through the nanopore, the individual nucleobases (117) can be captured by reader molecules attached to the electrodes to form a junction that facilitates the electron tunneling, creating electric signals for their recognition. The reader molecules interact with nucleobases via noncovalent bonds, such as hydrogen bonds,¹⁵ which is relatively week in aqueous solution. Accurate base identification or sequencing of a biopolymer, such as DNA, requires the movement of the molecule through the nanopore to be slow (at millisecond level) and controlled (with sub-nanometer precision). When a DNA molecule translocates through the nanopore without control, it is too fast (at microsecond level), so some individual nucleobases cannot be captured by the reader molecules, which results in deletion errors. Also, the fast movement of DNA does not give sufficient reaction time for the base-read molecule interaction to reach its equilibrium, which results in reading errors in the sequencing of nucleobases. Therefore, for the successful sequencing of a biopolymer using electron tunneling method, the slow and controlled movement of the biopolymer is an essential must-have condition.

The present invention provides a system that has a mechanism to slow down and control the movement of the target biopolymer passing through a nanopore, enabling the sequencing of the biopolymer using electron tunneling. In general, the said system (FIG. 1c) is consisted of an analysis stage equipped with a piezo actuator (100), a nanopore chip (140) with embedded tunneling nanogap (107) and reader molecules (110), a scan plate (130), a cis chamber (151) and a trans chamber (152), a DNA sample system (120) composed of a bead (108), a linker molecule (116) and the target DNA (109), and a voltage source for cross nanopore potential (115) and a voltage source for nanogap potential as well as signal measuring mechanism (111). The scan plate is placed substantially parallel to the nanopore chip. There are detailed descriptions about the analysis stage design and composition, the scan plate design and fabrication, as well as DNA sample construction methods in the previous patent applications, WO2017/075620, and PCT/US18/32399, which are included here in their entirety.

In some embodiments (ref. WO2017/075620), the biopolymer attaches to the scan plate directly through a chemical bond, either covalent or non-covalent, reversable or non-reversable, wherein the chemical bond is selected from the list comprising a biotin-streptavidin bond, an amide bond; a phosphodiester bond, ester bond, disulfide bond, imine bond, aldehyde bond, hydrogen bond, hydrophobic bonds, and a combination thereof.

In some embodiments, the system further comprises a controllable magnet, either an electromagnet or an adjustable magnet, or a group of magnet (ref. WO2017/075620). In FIG. 1c (A-C), the bead is a magnetic bead, made of paramagnetic, super-paramagnetic, ferromagnetic, or diamagnetic material. One end of the target single-stranded DNA molecule (ssDNA, 109) is attached to the magnetic bead (108) through a linker molecule (116) between them. The DNA sample with bead and linker molecule (120) is placed in the cis chamber (151). Under a biased potential through the voltage source 115, the free end of the ssDNA sample is pulled by electrophoretic force into a nanopore on the nanopore chip (140) and translocates through the nanopore into the trans chamber (152), while the other end is stopped at the nanopore entrance by the attached magnetic bead. By engaging or turning on the external magnet, the magnetic bead is attracted to the scan plate (130). By either applying strong magnetic force or through chemical bonding or other methods, the bead is tightly bound to the scan plate. Then, move the scan plate with the analysis stage (100) at a sub-nanometer precision (0.1 nm to 1 nm), and the DNA molecule will move through the nanopore at the same speed as the scan plate, and its bases can be read out by the reader molecules (110) one by one when they pass through the nanogap (107). For accurate base sensing by tunneling measurement, the base-reader interaction (residence) time is required to be 1 ms or longer. The longer the residence time, the more accurate the base sensing, however, the lower the sequencing throughput. So, generally, the preferred time is from 1 ms to 5 ms per base unit, and less preferably 0.1 ms to 100 ms per base unit. For ssDNA, the base unit size is about 0.34 nm and spans 0.7 nm when fully stretched, which requires the scan plate to be moved with a speed of about 0.1 μm/sec to 1 μm/sec, less preferably 0.005 μm/sec to 10 μm/sec.

In some embodiments (ref. PCT/US18/32399), in order to achieve strong localized magnetic force to hold the magnetic bead tightly upto the scan plate, a layer of micro-soft magnetic structures (102) are contructed on the surface of the scan plate (101), either standout structures or patterned holes filled with permalloy. The soft magnetic structure has a layout of a solitary structure, a grid array, a hexagonal array, a solitary strip, a linear array of strips across an area, a patterned array of clusters of structures, a random pattern of structures, etc. The soft magnetic structure may have the shape such as a circular cylinder, an oval cylinder, a rectangular block, a polygonal cylinder, a pyramid, an inverted pyramid, a cone, an inverted cone, an elongated shape, an irregular particle, a ring, etc. The size (diameter or width or equivalent dimension) ranges from 100 nm to 20 micrometer, preferably 1 micrometer to 5 micrometer. The center to center distance (or pitch) is usually 1 to 2 times the microstructure size. Except a permalloy, other nickel and ion alloys can also be used as the core of the soft magnetic structure, such as a nickel-iron-molybdenum alloy, a nickel-cobalt alloy, an iron-nickel-cobalt alloy, an iron-silicon alloy, a nickel and iron alloy with different percentages of nickel and iron, any number between 0% and 100%.

In some embodiments, the scan plate has micro-pillars or patterned areas or an array of micro-pillars or patterned areas for the attachment of the target DNA molecules or other biopolymers. The array of micro-pillars or patterned areas are substantially aligned with the nanopore or nanoslit array on the nanochip (ref. WO2017/075620).

In some embodiments, the target ssDNA molecule is attached to the scan plate by chemical bonding through a linker molecule without the bead. The target ssDNA molecule is ligated to the linker molecule and the linker molecule is attached to the scan plate. To identify or sequence the DNA, the scan plate is first lowered to allow the free end of the target ssDNA to enter the nanopore and translocate from the cis side to the trans side of the nanopore chip, and then move away from the nanopore. The target DNA can be sequenced when the ssDNA enters the nanopore and/or when it leaves the nanopore. It can be sequenced repeatly if needed to increase the accuracy.

In some embodiments, a double-stranded DNA or a single-stranded DNA, a polypeptide chain, a cellulose fiber or any flexible linear polymer, or the combination thereof, either natural, modified or synthesized, can used as the linker molecule (ref. WO2017/075620). A natural lambda DNA, which is about 48.5 kb, 16.5 micrometer long (double strand) or 34 micrometer long (single strand), is a good candidate for the linker molecule.

In some embodiment, a linker node, such as a non-magnetic bead or particle or a protein, is disposed between the linker molecule and the target DNA molecule and the linker node is configured to block the linker molecule from entering the nanopore to facilitate the alignment procedure (ref. WO2017/075620). The protein that can be used as a linker node includes but not limited to an antibody, an enzyme, a NeutrAvidin, a streptavidin, and an avidin. A linker node can be a polymer complex or particle or bead, a portion thereof, and a combination thereof.

In another embodiment, the nanopore is a nanoslit with the dimension of width in the range of 1 to 50 nm, preferably 2 to 20 nm, most preferably 2 to 5 nm, and the dimension of length 5 nm to 1 μm or no greater than the bead size, preferably 10 to 500 nm, most preferably 20 to 100 nm. The planar nanogap is built across the width of the nanoslit.

In one embodiment, this invention provides a detailed process for the fabrication of an electron tunneling nanopore (FIGS. 2A and 2B). A membrane layer (FIG. 2A, panel A, 202) is deposited on a base substrate (FIG. 2A, panel A, 201). The substrate 201 can be any material (such as Si-based, group III to V materials, or glass). Following the deposition, the substrate is etched from the backside to provide a supporting structure with a window (FIG. 2A, panel B, 203, and 204) for the tunneling nanopore device. A conductive layer (FIG. 2A, panel C, 205) is deposited as a bottom electrode on the 202 layer by a deposition technique including chemical vapor deposition (CVD), atomic layer deposition (ALD), physical vapor deposition (PVD), molecular vapor deposition (MVD), Electroplating, or Spin Coating etc., but not limited to those. The materials for the layer include but are not limited to metallic materials such as Au, Pt, Pd, W, Ti, Ta, Al, Ag, Cr, Cu, or conductive composite materials such as nitride compound (TiNx, TaNx) or oxide compound with or without doping. A various combination of sub-layers can be used as a multi-layer electrode to improve the adhesion and to control the conductivity of the electrode. In turn, an insulating layer (FIG. 2A, panel C, 206) is deposited by a technique of chemical vapor deposition (CVD), atomic layer deposition (ALD), physical vapor deposition (PVD), molecular vapor deposition (MVD), electroplating, or spin coating, etc., but not limited to those. The material can be any electrical insulator such as SiNx, SiOx, HfOx, Al₂O₃, and/or dielectrics used in the semiconductor industry. The insulating layer has a thickness of 2 to 5 nm, preferring to 3 to 4 nm. A 2^{nd} conductive layer (FIG. 2A, panel C, 207) is deposited as a top electrode by the same method as layer 205. A various combination of sub-layers can be used as a multi-layer electrode to improve the adhesion and control the conductivity of the electrode. A second electrically insulating layer (FIG. 2A, panel C, 208) is deposited on top of the conductive layer 207 as a cap layer to prevent the device from a short when exposed to an electrically conductive solution except for the exposed tunneling junction. A patterned etch mask is prepared to fabricate the tunneling junction embedded in nanopores using e-bam lithography on an e-beam resist or extreme ultraviolet lithography (EUV) on EUV resist, which serves as a etch mask after development. The resist mask is used as a pattern transfer (FIG. 2B, panel D, 209, 210) for a following multi-layered hard mask that serves as a final patterned etch mask. A reactive ion etching (RIE), plasma dry etching, focused ion beam (FIB), focused electron beam (FEB), or ion beam etching (IBE) is performed to etch through the cap layer (208), top electrode (207), insulating layer (206), bottom electrode (205), and the membrane (202). A stacked tunneling nanogap embedded in a nanopore (FIG. 2B, panel E, 211) is created after removing the etch residues and the etch mask. The nanopore is sized with a diameter of 1 to 50 nm, preferably 2 to 20 nm, most preferably 2 to 5 nm.

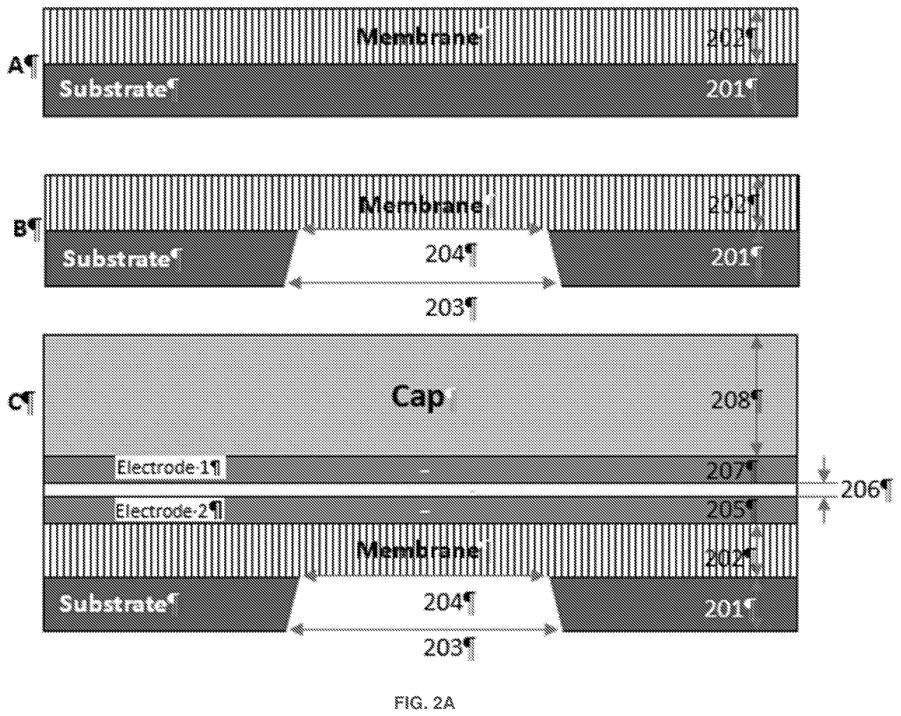

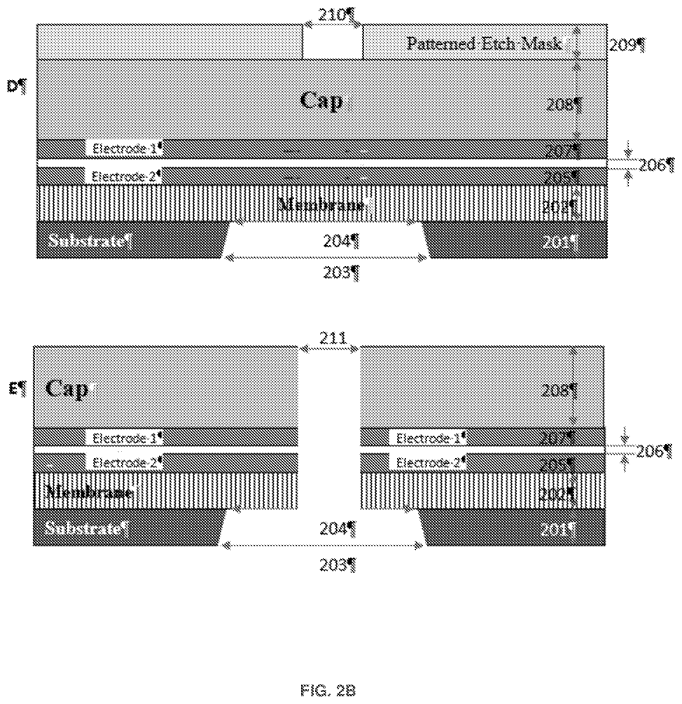

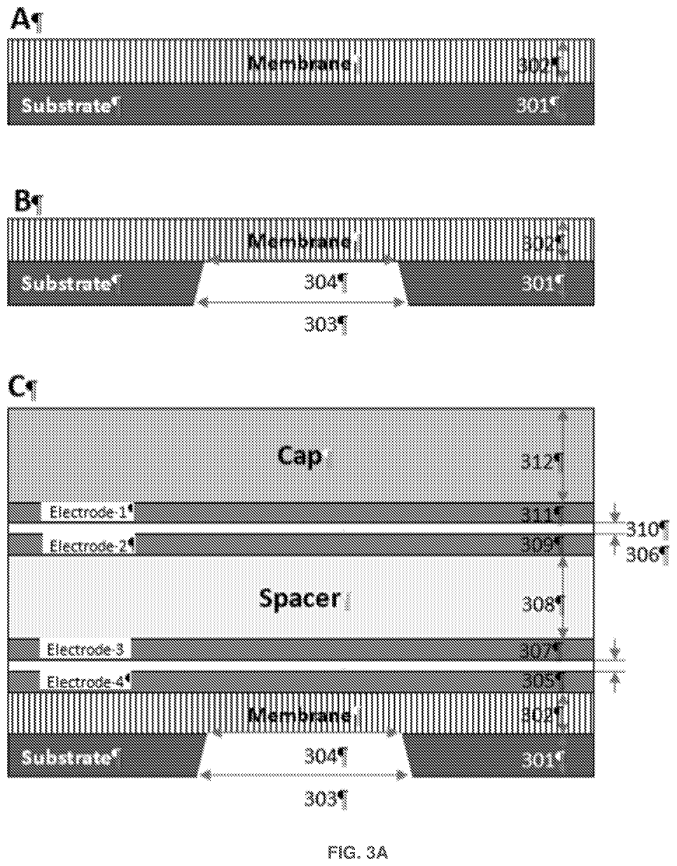

In another embodiment, this invention provides a process to fabricate a device with two tunneling gaps (four electrodes) with the same type or different types of reader molecules embedded in a nanopore (FIGS. 3A and B) for reading polymeric sequences repeatedly, which increases the sequencing accuracy. The tunneling gaps are separated by a spacer (FIG. 3A, panel C, 308). The spacer can be any electrically insulating material such as SiNx, SiOx, HfOx, Al₂O₃, and/or dielectric materials used in the semiconductor industry. The deposition of the spacer is performed with a technique of chemical vapor deposition (CVD), atomic layer deposition (ALD), physical vapor deposition (PVD), molecular vapor deposition (MVD), electroplating, or spin coating, etc., but not limited to those. The other process for the fabrication of the device follows those described in [0027]. In considering a higher aspect ratio of the tunneling junction compared to a single tunneling nanogap, a multi-layer hard mask approach is preferable, using a patterned resist as a pattern transfer mask, as mentioned in [0027].

In one embodiment, this invention provides a detailed process for the fabrication of a device composed of a planar tunneling nanogap embedded in a nanopore (FIGS. 4A and B). An insulating layer (FIG. 4, panel A, 402) is deposited on a base substrate (FIG. 4, panel A, 401). The substrate 401 can be any material (such as Si-based, group III to V, or glass). Following the deposition, the substrate is etched from the backside to provide a supporting structure with an open window (FIG. 4, panel A, 403, and 404) for the tunneling nanopore device. In turn, an electrically conductive layer (FIG. 4, panel A, 405) is deposited on the insulating layer 402. The conductive layer can be a multi-layer structure to enhance the adhesion and to control the desired conductivity. An adhesive layer is deposited to either act as a second conductive electrode or insulating/protective layer (FIG. 4, panel A, 409) if needed. A reactive ion etching (RIE), plasma dry etching, focused ion beam (FIB), focused electron beam (FEB), or ion beam etching (IBE) is performed to fabricate a nanowire. After etching and removing of the resist residue and cleaning the substrate, an electrically insulating materials (FIG. 4, panel A, 406) is deposited as a capping layer to protect the electrode when exposing in a conductive solution except for the nanogap cross-section. A non-conductive layer (FIG. 4, panel A, 410) as an adhesion promoter is deposited for the photoresist if needed. Photoresist (FIG. 4, panel A, 407) can be patterned using electron beam lithography (EBL) or extreme ultraviolet lithography (EUV) to define the nanogap cross the nanowire (FIG. 4, panel A, 408). This patterned resist can act as a direct etch mask or can be used for the pattern transfer to fabricate any hard mask. The gap pattern is crossing the nanowire pattern. The top of the sacrificial layer (410) or the cap insulating layer (406) can be seen between the gap. A reactive ion etching (RIE), plasma dry etching, focused ion beam (FIB), focused electron beam (FEB), or ion beam etching (IBE) is performed to etch through the layers to form a nanopore with an embedded tunneling nanogap as shown in FIG. 4B. After strip/cleaning, the topmost layer can be the sacrificial layer (410) or the cap insulating layer (406).

In some embodiments, the reader molecules are attached to those electrodes that form a tunneling nanogap to interact with an individual base unit of a polymer for their identification. The said interaction is hydrogen bonding, stacking, electrostatic, or other noncovalent interactions.

In some embodiments, the reader molecules disclosed in the prior arts, including 1.8-Napthyridine derivatives and imidazole-carboxamide derivatives (U.S. Pat. No. 8,628,649), benzamide (U.S. Pat. No. 9,140,682), triazole-carboxamide derivatives (U.S. Pat. No. 10,336,713), benzimidazole-2-carboxamide (US 2016/0108002), pyrene derivatives (US 2019/0195856), are used to read the basic units of bio- and synthetic polymers by electron tunneling.

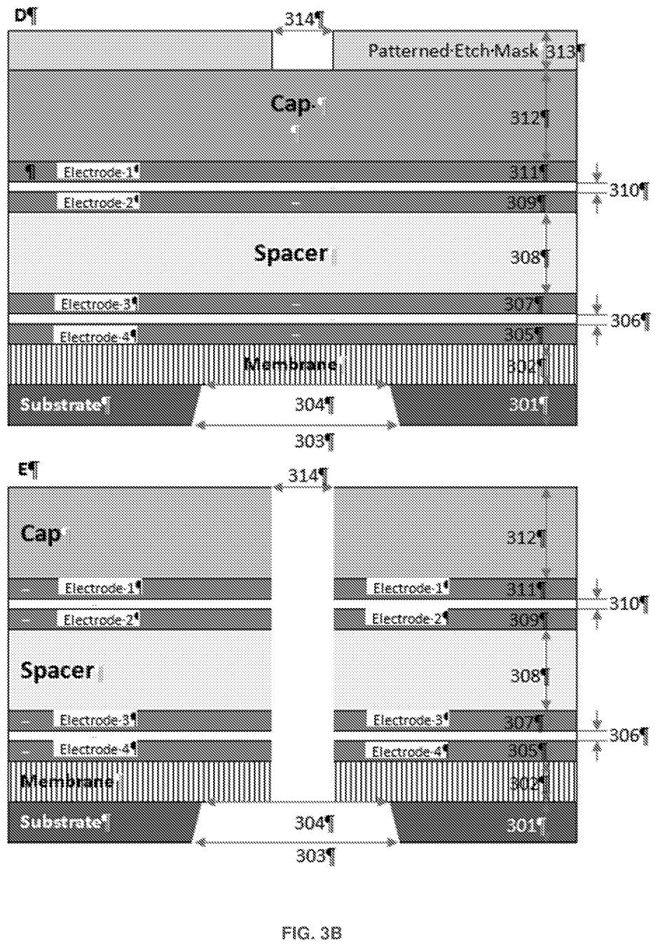

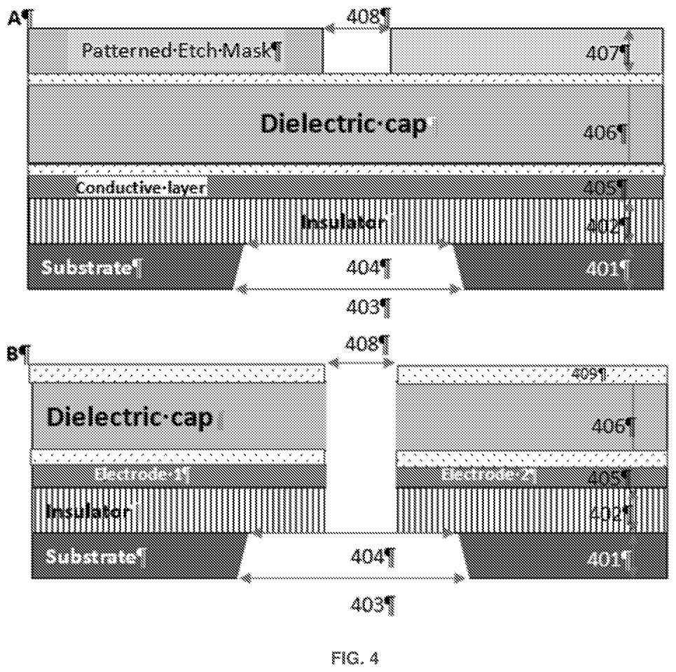

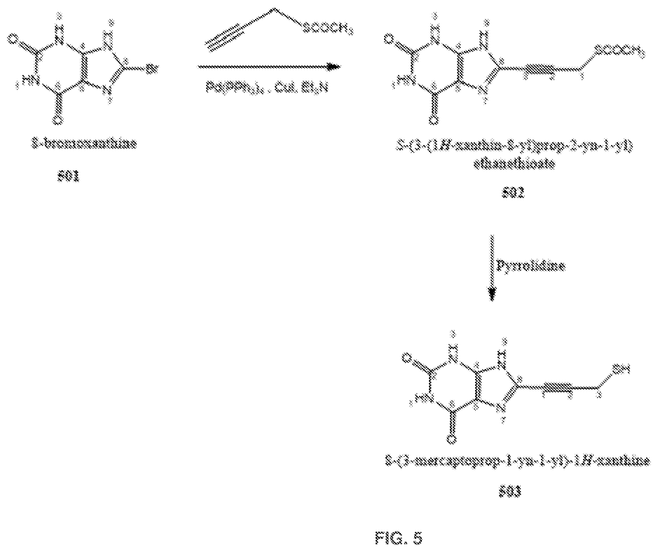

In one embodiment, this invention exploits xanthine as a reader molecule (FIG. 5, 503) for reading sequences of biopolymers including naturally occurring nucleic acids, proteins, peptides, and polysaccharides as well as those synthetic nucleic acids such as XNA and nucleic acids analogs such as peptide nucleic acid (PNA), re-engineered proteins with unnatural amino acids, modified peptides. The compound 503 is synthesized following a route, as shown in FIG. 5, starting from 8-bromoxanthine (501).

In one embodiment, molecular modeling indicates that the reader molecule 503 interacts with DNA bases through hydrogen bonding to form different triplet complexes (FIG. 6) with two sulfur atoms fixed at a distance of 2.8 nm so the tunneling currents would flow through these structures differently, which are used as signatures for individual nucleosides. When a DNA molecule translocates through the tunneling nanogap, its sequence can be read out by the tunneling signatures. Given that the Au—S bond has a length of 2.156 Å,¹⁶ a nanogap with a size of ˜3.2 nm is practical for the tunneling sequencing. That provides a manufacturing advantage for the fabrication of the tunneling nanopore devices compared to those tunneling junctions with their gap sizes around 2.5 nm.

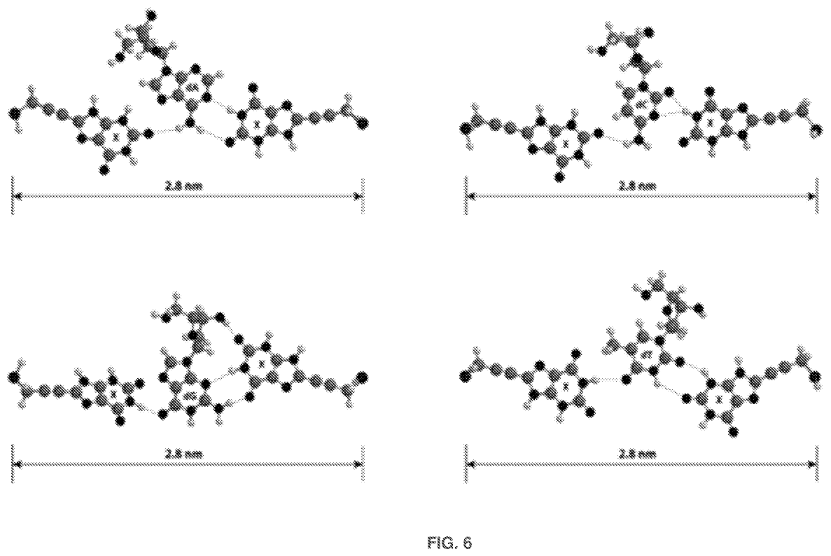

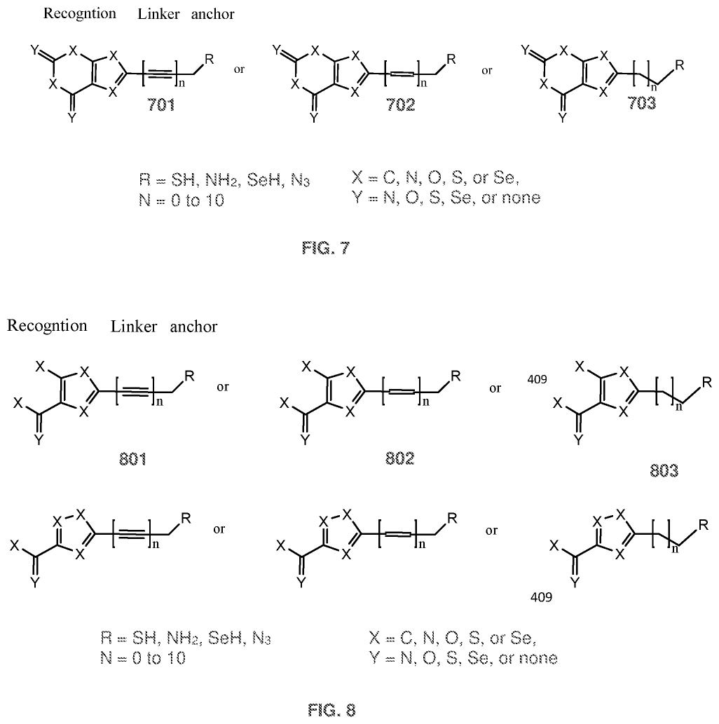

In some embodiments, the structure of reader molecules can be described as a general form, as expressed in FIG. 7. It is composed of a recognition moiety that interacts with the monomers of a polymer through noncovalent forces and an anchor that is used to fix the recognition moiety on electrodes, both of which are connected through a linker 701, 702, or 703, which allows different electron tunnelings.

In some embodiments, the invention provides a series of reader molecules with a smaller size derived from the xanthine reader molecules (FIG. 8). These reader molecules are suitable to the tunneling nanogaps, preferably with their sizes around 2.5 nm. Their linkers 801, 802, or 803 are equivalent to those corresponding 701, 702, or 703, respectively.

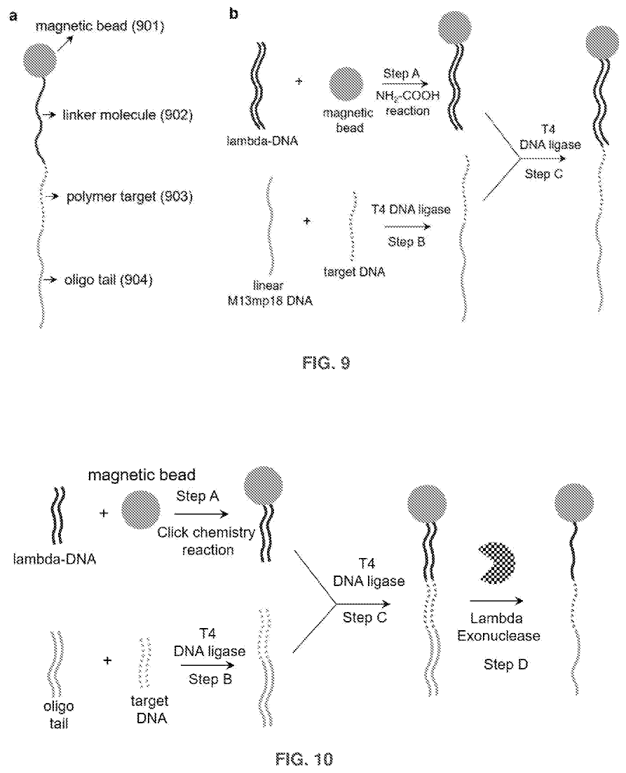

In some embodiments, this invention provides a method to prepare a biopolymer sample construct for its analysis by the said tunneling nanopore device. As shown in FIG. 9, the construct has a polymeric target (FIG. 9, 903) attached to a magnetic bead (901) through a molecular linker (902), tailed with an oligonucleotide to prevent the target from jumping out of the nanopore. The size of magnetic beads ranges from 50 nm to 20 μm, preferably 1 μm to 3 μm, and the molecular linker comprises negatively charged DNA (either single-stranded or double-stranded) and RNA, neutral polyethylene glycol (PEG), or positively charged polyethyleneimine under the physiological conditions, but not limited to them. The polymeric target is naturally occurring DNA, RNA, proteins, polysaccharides, and their modified artificial counterparts. The oligo tail is composed of negatively charged DNA (either single-stranded or double-stranded) and RNA, neutral polyethylene glycol (PEG), or positively charged polyethyleneimine under the physiological conditions, but not limited to them.

This invention provides examples for the preparation of DNA constructs. One example (delineated in FIG. 9b) is a DNA construct. First, λ-DNA functionalized with an amine at its one end, used as a linker molecule, is attached to a magnetic bead functionalized with carboxylate via an amidation reaction (Step A).¹⁷ In other embodiments, this step employs different chemical reactions such as azide-alkyne cycloaddition, maleimide-thiol coupling, etc. In parallel, a DNA target is ligated to a linear M13mp18 DNA tail via a T4 DNA ligase (Step B).¹⁸ In some embodiments, this step is finished via a chemical reaction or other enzymatic ligation such as T7, Taq, etc. Then, the target-tail conjugate is linked to the λ-DNA linker attached to the magnetic bead through the T4 DNA ligation (Step C). In some embodiments, this step is completed by another ligase such as T7, Taq, etc., or chemical reactions such as, but not limited to amine-carboxylate, thiol-maleimide, or click coupling.^{19,20} In some embodiments, the sample construct is prepared firstly by connecting a linker molecule, a target molecule, and a linker molecule to form a linker-target-tail conjugate that is then attached to a magnetic bead.

Another example is preparing a DNA construct starting with a double-stranded DNA sample, using the linear pUC19 vector as a tail (FIG. 10). First, λ-DNA is attached to magnetic beads via an azide-alkyne click reaction (Step A). In parallel, the double-stranded DNA target is ligated to a double-stranded DNA tail through the T4 DNA ligation (Step B). In some embodiments, different ligases such as T3, T7, Taq are be used depending on targets to be ligated. The target-tail conjugate is then conjugated to the λ-DNA on the magnetic bead (Step C). In turn, the double-stranded DNA construct on the magnetic bead is digested by λ-exonuclease²¹ to obtain a single-stranded DNA construct on the magnetic bead (Step D). In another embodiment, the ligated double-stranded linker-target-tail complex is prepared and digested before the attachment to the magnetic bead.

In some embodiments, a nanochip containing an array of nanopores between 100 to 100 million, preferably between 1,000 to 1 million, is made in order to satisfy the throughput requirements of biopolymer sensing or sequencing.

In some embodiments, an array of nanopore devices on one chip is divided into multiple regions or modules and the signals are read out separately from one region to other regions by separate signal recording units in order to overcome the bandwidth and sampling frequency limits of a single recording unit.

### GENERAL REMARKS

All publications, patents, and other documents mentioned herein are incorporated by reference in their entirety. Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of the applicant's general inventive concept.

### REFERENCES

1 Metzker, M. L. Emerging technologies in DNA sequencing. Genome Res 15, 1767-1776, (2005).
2 Goodwin, S., McPherson, J. D. & McCombie, W. R. Coming of age: ten years of next-generation sequencing technologies. Nature reviews. Genetics 17, 333-351, (2016).
3 Treangen, T. J. & Salzberg, S. L. Repetitive DNA and next-generation sequencing: computational challenges and solutions. Nature reviews. Genetics 13, 36-46, (2012).
4 Rhoads, A. & Au, K. F. PacBio Sequencing and Its Applications. Genomics Proteomics Bioinformatics 13, 278-289, (2015).
5 Gordon, D. et al. Long-read sequence assembly of the gorilla genome. Science 352, aae0344-0341, (2016).
6 Mukhopadhyay, R. DNA sequencers: the next generation. Analytical Chemistry 81, 1736-1740 (2009).
7 Deamer, D., Akeson, M. & Branton, D. Three decades of nanopore sequencing. Nat Biotechnol 34, 518-524, (2016).
8 Jain, M. et al. Improved data analysis for the MinION nanopore sequencer. Nature methods 12, 351-356, (2015).
9 Manrao, E. A. et al. Reading DNA at single-nucleotide resolution with a mutant MspA nanopore and phi29 DNA polymerase. Nat Biotechnol 30, 349-353, (2012).
10 Laszlo, A. H. et al. Decoding long nanopore sequencing reads of natural DNA. Nature Biotechnology 32, 829-833, (2014).
11 Lindsay, S. The promises and challenges of solid-state sequencing. Nat Nanotechnol 11, 109-111, (2016).
12 Lagerqvist, J., Zwolak, M. & Ventra, M. D. Fast DNA Sequencing via Transverse Electronic Transport. Nano Lett. 6, 779-782 (2006).
13 Di Ventra, M. & Taniguchi, M. Decoding DNA, RNA and peptides with quantum tunnelling. Nat Nanotechnol 11, 117-126, (2016).
14 Biswas, S. et al. Universal Readers Based on Hydrogen Bonding or pi-pi Stacking for Identification of DNA Nucleotides in Electron Tunnel Junctions. ACS Nano 10, 11304-11316, (2016).
15 Im, J., Sen, S., Lindsay, S. & Zhang, P. Recognition Tunneling of Canonical and Modified RNA Nucleotides for Their Identification with the Aid of Machine Learning. ACS Nano 12, 7067-7075, (2018).
16 Kokkin, D. L. et al. Au—S Bonding Revealed from the Characterization of Diatomic Gold Sulfide, AuS. The Journal of Physical Chemistry A 119, 11659-11667, (2015).
17 Sehgal, D. & Vijay, I. K. A Method for the High Efficiency of Water-Soluble Carbodiimide-Mediated Amidation. Analytical Biochemistry 218, 87-91, (1994).
18 Green, M. R. & Sambrook, J. Molecular Cloning: A Laboratory Manual. 4th edn, Vol. 1 235 (Cold Spring Harbor Laboratory Press, 2012).
19 El-Sagheer, A. H. & Brown, T. Click Nucleic Acid Ligation: Applications in Biology and Nanotechnology. Accounts of Chemical Research 45, 1258-1267, (2012).
20 El-Sagheer, A. H. & Brown, T. Click chemistry with DNA. Chemical Society Reviews 39, 1388-1405, (2010).
21 Little, J. W. An Exonuclease Induced by Bacteriophage λ: II. Nature of the Enzymatic Reaction. Journal of Biological Chemistry 242, 679-686 (1967).

What is claimed:

1. A system for electronic identification and sequencing of a biopolymer comprising:
(a) a substrate positioned between a cis space and a trans space, wherein the substrate comprises at least one conductive layer and at least one insulation layer;
(b) a nano-opening in the substrate, wherein at least a portion of the biopolymer can pass through from the cis space to the trans space;
(c) a nanogap formed by a first electrode and a second electrode embedded in the nano-opening;
(d) at least one pair of first and second reader molecules attached to the electrodes, wherein the first reader molecule is attached to the first electrode and the second reader molecule is attached to the second electrode, and wherein the pair of first and second reader molecules are configured to interact with the biopolymer for conducting electron tunneling current;
(e) a scan plate located in the cis space to which directly or indirectly a first end of the biopolymer is attached;
(f) an actuator for controlling a distance between the substrate and the scan plate such that the distance can be controlled with nanometer precision;
(g) a first bias source for applying a bias voltage between the cis space and the trans space to direct a second end of the biopolymer to enter the nano-opening;
(h) a second bias source for applying a bias voltage between the first and the second electrodes at the nanogap embedded in the nano-opening to facilitate electron tunneling measurement; and
(i) a software is configured to identify the biopolymer or a base unit of the biopolymer based on an electron tunneling signal or a plurality of electron signals sensed through the reader molecules.

2. The system of claim 1, wherein the biopolymer is selected from the group consisting of a DNA, an RNA, an XNA, a PNA, a protein, a carbohydrate, a sugar, a nucleic acid oligo, a peptide, a polysaccharide, either natural, modified, or synthetic, and a combination thereof.

3. The system of claim 1, wherein the nano-opening comprises either a nanopore or a nano-slit, either natural (biological), synthetic, or a combination thereof.

4. The system of claim 3, wherein the nanopore is substantially circular in shape with a diameter from about 2 nm to about 50 nm, and the nano-slit is substantially rectangular in shape with a length from about 5 nm to about 1 micrometer and a width from about 2 nm to about 50 nm.

5. The system of claim 3, wherein the nanopore is substantially circular in shape with a diameter from about 2 nm to about 5 nm, and the nano-slit is substantially rectangular in shape with a length from about 20 nm to about 100 nm and a width from about 2 nm to 5 nm.

6. The system of claim 1, wherein the nano-opening comprises an array of about 100 to about 1 million nano-openings, wherein each nano-opening comprises a nanogap embedded.

7. The system of claim 1, wherein the nanogap is a planar nanogap, and wherein the first electrode and the second electrode are in the same plane with their end surfaces being exposed to the nano-opening facing each other, separated by a distance substantially equal to the size of the nano-opening.

8. The system of claim 1, wherein the nanogap is a vertical nanogap, wherein the first electrode and the second electrode are in different planes overlapping each other with an insulation layer in between, and wherein a thickness of the insulation layer is between about 2 nm to about 5 nm, preferably about 3 nm to about 4 nm, and the nano-opening cuts through both the electrodes and the insulation layer.

9. The system of claim 1, comprising two pairs of electrodes embedded in the nano-opening, forming two nanogaps, wherein one pair is near the top of the nano-opening, and another pair is near the bottom of the nano-opening, with an insulating spacer layer separating them.

10. The system of claim 1, wherein the electrodes comprise a material selected from the group consisting of a metallic material comprising Au, Pt, Pd, W, Ti, Ta, Al, Ag, Cr, or Cu; a conductive composite material comprising TiNx, or TaNx; an oxide compound with or without doping; and a combination thereof; and wherein the insulation layer comprises a material selected from the group consisting of a dielectric insulating material comprising SiNx, SiOx, HfOx, or Al2O3; and a combination thereof.

11. The system of claim 1, comprising a plurality of reader molecules on each electrode, and wherein the reader molecule on one electrode does not physically touch any of the reader molecules on the opposite electrode.

12. The system of claim 1, wherein the reader molecule is selected from the group consisting of the following:
(a) a 1.8-Napthyridine derivative;
(b) an imidazole-carboxamide derivative;
(c) a benzamide;
(d) a triazole-carboxamide derivative;
(e) a benzimidazole-2-carboxamide;
(f) a pyrene derivative;
(g) a xanthine; and
(h) a combination of any of the above.

13. The system of claim 1, wherein the reader molecule comprises a xanthine, either natural, modified or synthesized; and a combination thereof.

14. The system of claim 1, wherein the reader molecule comprises a linker and an anchor, wherein the anchor attaches the reader molecule to the electrode, and the linker is between the anchor and a recognition moiety of the reader molecule.

15. The system of claim 1, wherein the scan plate and the substrate are substantially parallel, and the distance between the substrate and the scan plate can be adjusted at a rate of about 0.1 ms to about 100 ms per base unit of the biopolymer or about 0.005 micrometer/sec to about 10 micrometer/sec.

16. The system of claim 1, wherein the scan plate and the substrate are substantially parallel, and the distance between the substrate and the scan plate can be adjusted at a rate of 1 ms to 5 ms per base unit of the biopolymer or about 0.1 micrometer/sec to about 1 micrometer/sec.

17. The system of claim 1, wherein the actuator comprises a precision linear motion stage driven by a piezo-electric drive with nanometer or sub-nanometer precision.

18. The system of claim 1, wherein the scan plate contains a micro-structure or a micro-patterned area or an array of micro-structures or an array of micro-patterned areas, onto which directly or indirectly a first end of the biopolymer is attached.

19. The system of claim 18, wherein the micro-structure or the micro-patterned area comprises a size, such as a diameter or a length/width or an equivalent dimension, of about 0.1 micrometer to about 20 micrometer.

20. The system of claim 18, wherein the micro-structure or the micro patterned area comprises a soft magnetic material selected from the group consisting of a permalloy, a nickel-iron-molybdenum alloy, a nickel-iron alloy, a substantially pure nickel, a substantially pure iron, a nickel-cobalt alloy, an iron-nickel-cobalt alloy, and an iron-silicon alloy, and a combination thereof.

21. The system of claim 1, further comprising a linker molecule, wherein the linker molecule is attached to the first end of the biopolymer at one end and attached to the scan plate at the other end.

22. The system of claim 21, wherein the linker molecule is selected from the group consisting of a single stranded nucleic acid, a double stranded nucleic acid, a polypeptide chain, a cellulose fiber or any flexible linear polymer, either natural, modified or synthesized, and a combination thereof.

23. The system of claim 21, wherein the linker molecule is a lambda DNA, single or double stranded, either natural, modified or synthesized, and a combination thereof.

24. The system of claim 21, further comprising a magnet and a magnetic bead, wherein the linker molecule is attached to the first end of the biopolymer at one end and attached to the magnetic bead at the other end, and the magnet is configured to attract the magnetic bead towards the scan plate and to hold the magnetic bead against the scan plate so that the magnetic bead can move with the scan plate, and wherein the magnet comprises an electromagnet, an adjustable permanent magnet, a group of magnets, or a combination thereof.

25. The system of claim 24, wherein the size of the magnetic bead ranges in diameter from about 50 nm to about 20 micrometer, preferably about 1 micrometer to about 3 micrometer.

26. The system of claim 1, further comprising an oligo tail, wherein the oligo tail is attached to a second end of the biopolymer.

27. The system of claim 26, wherein the oligo tail is selected from the group consisting of a single stranded DNA or RNA, a double stranded DNA or RNA, a polyethylene glycol, a polyethyleneimine, and a combination thereof.

28. The system of claim 26, wherein the oligo tail comprises a linear M13mp18 DNA or a linear pUC19 vector.

29. The system of claim 21 or 26, wherein the linker molecule and the oligo tail are attached to the biopolymer by ligation.

30. The system of claim 1, 18, 21 or 24, wherein the attachment of the biopolymer to the scan plate, the attachment of the linker molecule to the scan plate and the magnetic bead, and the attachment of the reader molecule to the electrode, are through a covalent chemical bond.

31. A method for electronic identification and sequencing of a biopolymer comprising:
(a) providing a substrate with a nano-opening and a nanogap formed by a first electrode and a second electrode embedded in the nano-opening;
(b) attaching at least one pair of first and second reader molecules to the electrodes, the first reader molecule is attached to the first electrode and the second reader molecule is attached to the second electrode, that can interact with the biopolymer for conducting electron tunneling current;
(c) positioning the substrate between a cis space and a trans space, wherein at least a portion of the biopolymer can pass from the cis space to the trans space through the nano-opening;
(d) providing a scan plate and an actuator with nanometer precision;
(e) placing the scan plate in the cis space substantially parallel to the substrate;
(f) attaching directly or indirectly a first end of the biopolymer to the scan plate;
(g) providing a first bias source for applying a bias voltage between the cis space and the trans space to direct a second end of the biopolymer to enter the nano-opening;
(h) providing a second bias source for applying a bias voltage between the first and the second electrodes at the nanogap embedded in the nano-opening to facilitate electron tunneling measurement;
(i) adjusting the distance between the substrate and the scan plate by either moving the substrate or the scan plate or both with an actuator; wherein the biopolymer moves through the nanogap and interacts with the reader molecules;
(j) recording the electron tunneling signal through the reader molecule;
(k) identifying the biopolymer or a base unit of the biopolymer based on the signal.

32. The method of claim 31, wherein the biopolymer is selected from the group consisting of a DNA, an RNA, an XNA, a PNA, a protein, a carbohydrate, a sugar, a nucleic acid oligo, a peptide, a polysaccharide, either natural, modified, or synthetic, and the combination thereof.

33. The method of claim 31, wherein the nano-opening is either a nanopore or a nano-slit, either natural (biological), synthetic, or a combination thereof.

34. The method of claim 33, wherein the nanopore is substantially circular in shape with a diameter from about 2 nm to about 50 nm, and the nano-slit is substantially rectangular in shape with a length from about 5 nm to about 1 micrometer and a width from about 2 nm to about 50 nm.

35. The method of claim 33, wherein the nanopore is substantially circular in shape with a diameter from about 2 nm to about 5 nm, and the nano-slit is substantially rectangular in shape with a length from about 20 nm to about 100 nm and a width from about 2 nm to about 5 nm.

36. The method of claim 31, wherein the nano-opening is an array of about 100 to about 1 million nano-openings, each nano-opening comprises a nanogap embedded.

37. The method of claim 31, wherein the nanogap is a planar nanogap, wherein the first electrode and the second electrode are in the same plane with their end surfaces being exposed to the nano-opening facing each other, separated by a distance substantially equal to the nano-opening size.

38. The method of claim 31, wherein the nanogap is a vertical nanogap, wherein the first electrode and the second electrode are in different planes overlapping each other with an insulation layer in between, wherein a thickness of the insulation layer is between about 2 nm to about 5 nm, preferably 3 nm to 4 nm, and the nano-opening cuts through both the electrodes and the insulation layer.

39. The method of claim 31, comprising two pairs of electrodes embedded in the nano-opening, forming two nanogaps, one pair is near the top of the nano-opening, and another pair is near the bottom of the nano-opening, with an insulating spacer layer separating them.

40. The method of claim 31, wherein the electrodes comprise a material selected from the group consisting of a metallic material, comprising Au, Pt, Pd, W, Ti, Ta, Al, Ag, Cr, or Cu; a conductive composite material, comprising TiNx, or TaNx; an oxide compound with or without doping; and a combination thereof; and wherein the insulation layer comprises a material selected from the group consisting of a dielectric insulating material, comprising SiNx, SiOx, HfOx, Al₂O₃, and a combination thereof.

41. The method of claim 31, comprising a plurality of reader molecules attached to each electrode, wherein a reader molecule on one electrode does not physically touch any of the reader molecules on the opposite electrode.

42. The method of claim 31, wherein the reader molecule is selected from the group consisting of the following:
(a) a 1.8-Napthyridine derivative;
(b) a imidazole-carboxamide derivative;
(c) a benzamide;
(d) a triazole-carboxamide derivative;
(e) a benzimidazole-2-carboxamide;
(f) a pyrene derivative;
(g) a xanthine; and
(h) a combination of any of the above.

43. The method of claim 31, wherein the reader molecule comprises a xanthine, either natural, modified or synthesized or a combination thereof.

44. The method of claim 31, wherein the reader molecule comprises a linker and an anchor, wherein the anchor attaches the reader molecule to the electrode, and the linker is between the anchor and a recognition moiety of the reader molecule.

45. The method of claim 31, wherein the distance between the substrate and the scan plate can be adjusted at a rate of about 0.1 ms to about 100 ms per base unit of the biopolymer or about 0.005 micrometer/sec to about 10 micrometer/sec.

46. The method of claim 31, wherein the distance between the substrate and the scan plate can be adjusted at a rate of about 1 ms to about 5 ms per base unit of the biopolymer or about 0.1 micrometer/sec to about 1 micrometer/sec.

47. The method of claim 31, wherein the actuator comprises a precision linear motion stage driven by a piezo-electric drive with nanometer or sub-nanometer precision.

48. The method of claim 31, wherein the scan plate comprises a micro-structure or a micro-patterned area or an array of micro-structures or an array micro-patterned areas, onto which directly or indirectly a first end of the biopolymer can be attached.

49. The method of claim 48, wherein the micro-structure or the micro-patterned area has a size, such as diameter or length/width or equivalent dimension, of about 0.1 micrometer to about 20 micrometer.

50. The method of claim 48, wherein the micro-structure or the micro patterned area is made of a soft magnetic material selected from the group consisting of a permalloy, a nickel-iron-molybdenum alloy, a nickel-iron alloy, a substantially pure nickel, a substantially pure iron, a nickel-cobalt alloy, an iron-nickel-cobalt alloy, an iron-silicon alloy, and a combination thereof.

51. The method of claim 31, further comprising providing a linker molecule, wherein the linker molecule is attached to the first end of the biopolymer at one end and attached to the scan plate at the other end.

52. The method of claim 51, wherein the linker molecule is selected from the group consisting of a single stranded nucleic acid, a double stranded nucleic acid, a polypeptide chain, a cellulose fiber or any flexible linear polymer, either natural, modified or synthesized, and a combination thereof.

53. The method of claim 51, wherein the linker molecule is a lambda DNA, single or double stranded, either natural, modified or synthesized.

54. The method of claim 51, further comprising providing a magnet and a magnetic bead, wherein the linker molecule is attached to the first end of the biopolymer at one end and attached to the magnetic bead at the other end, and the magnet is configured to attract the magnetic bead towards to scan plate and to hold the magnetic bead against the scan plate so that it can move with the scan plate, wherein the magnet comprising an electromagnet, an adjustable permanent magnet, a group of magnets, or a combination thereof.

55. The method of claim 54, wherein the size of the magnetic bead ranges in diameter from about 50 nm to 20 micrometer, preferably 1 micrometer to 3 micrometer.

56. The method of claim 31, further comprising attaching an oligo tail to a second end of the biopolymer.

57. The method of claim 56, wherein the oligo tail is selected from the group consisting of a single stranded DNA or RNA, a double stranded DNA or RNA, a polyethylene glycol, a polyethyleneimine, and a combination thereof.

58. The method of claim 56, wherein the oligo tail is a linear M13mp18 DNA or a linear pUC19 vector.

59. The method of claim 51 or 56, wherein the linker molecule and the oligo tail are attached to the biopolymer by ligation.

60. The method of claim 31, 48, 51 or 54, wherein the attachment of the biopolymer to the scan plate, the attachment of the linker molecule to the scan plate and the magnetic bead, and the attachment of the reader molecule to the electrode, are through a covalent chemical bond.
